(19) United States
(12) Patent Application Publication
(10) Pub. No.: US 2023/0317279 A1
(43) Pub. Date: Oct. 5, 2023

(54) METHOD AND SYSTEM FOR MEDICAL DIAGNOSIS USING GRAPH EMBEDDINGS

(71) Applicant: Quantiphi Inc, Marlborough, MA (US)

(72) Inventors: Asif Hasan, Marlborough, MA (US); Vishal Vaddina, Mumbai (IN)

(21) Appl. No.: 17/711,009

(22) Filed: Mar. 31, 2022

(51) Int. Cl. G16H 50/20; G16H 10/60

(52) U.S. Cl. CPC G16H 50/20; G16H 10/60

(57) ABSTRACT

Disclosed is an AI-based method for medical diagnosis, comprising: obtaining medical data from data source(s); generating a Directed Acyclic Graph (DAG) (204) using medical data, wherein nodes of DAG represent at least set of symptoms and set of medical conditions, and wherein directed edges between nodes represent relations between nodes; generating graph embeddings for nodes of DAG using graph-embedding technique; receiving input indicative of person's health, wherein input comprises set of symptoms (202); extracting set of test graph embeddings corresponding to test set of symptoms from graph embeddings; and processing test graph embeddings using neural network (206) for obtaining multi-label classification (208) of test set of symptoms matched to test set of medical conditions as labels, wherein given label is probabilistically indicative of presence of medical condition.

### TECHNICAL FIELD

The present disclosure relates to artificial intelligence-based methods for medical diagnosis. Moreover, the present disclosure also relates to artificial intelligence-based systems for medical diagnosis.

### BACKGROUND

In the recent years, amounts of data powering the healthcare industry and its systems have vastly grown, leading to an exponential increase in data being generated and stored in almost every sector. Such data includes both: unstructured data (for example, raw text in documents, articles, medical journals, etc.), and structured data (for example, tables, electronic health records, HTML, etc.). Organizing such vast amounts of data and utilising the same to solve real-world problems is rewarding as well as challenging. Diagnosis or prognosis of a medical condition depends on various symptoms being faced, and a variety of other external factors in addition to the symptoms, for example, demographic attributes (for example, age, gender, geographic location, etc.), medical history (for example, existing conditions, ongoing treatments and analysis from historic medical tests, etc.) and more.

Presently, there exist a few symptom-checkers that are available online (i.e., over the Internet), but most of these systems are non-intelligence based and thereby lack the ability of learning new information and upscaling. Nowadays, machine learning systems are increasingly being employed to diagnose the medical condition, depending on the symptoms. Such systems traditionally utilise one-hot encodings (i.e., representing a vector with 1 for a current input token and 0s for the rest), which is not only a sparse representation, but also does not account for any actual meaning of the words (i.e., symptoms), lengths and/or inter-relations of the same. For example, word ‘high temperature’ would not be understood to be related to word ‘fever’, even though they are synonymous. One-hot encoding is not an ideal way of representing symptoms and systems built using such encoding have a low diagnostic accuracy. More recently, deep learning systems are being employed to diagnose the medical condition. Such systems represent the symptoms using dense representations which are usually learned by a language model. Although these systems are superior to the traditional machine learning systems and have an improved performance compared thereto, representations for such systems rely on textual entities (i.e., words or sentences) and cannot attain meaningful embeddings while taking the external factors into account. As an example, deep learning-based systems may employ a Transformer-based Universal Sentence Encoder (USE) that provides a dense-representation of words or sentences. In USE, definitions and other available textual descriptions of symptoms and diseases are used for enabling the deep learning-based systems to learn latent relations between a combination of symptoms and medical conditions. However, none of the presently available solutions factor in the above-mentioned external factors (for example, demographic attributes, medical history, and more), which are important for accurately diagnosing the medical condition. As a result, the presently available systems have poor accuracy for medical diagnosis, which is undesirable.

Therefore, in light of the foregoing discussion, there exists a need to overcome the drawbacks associated with conventional systems and methods for medical diagnosis.

### SUMMARY

The present disclosure seeks to provide an artificial intelligence-based method for medical diagnosis. The present disclosure also seeks to provide an artificial intelligence-based system for medical diagnosis. An aim of the present disclosure is to provide a solution that overcomes at least partially the problems encountered in prior art.

In one aspect, an embodiment of the present disclosure provides an artificial intelligence (AI)-based method for medical diagnosis, the method comprising:
obtaining medical data from at least one data source;
generating a Directed Acyclic Graph (DAG) using the medical data, wherein nodes of the DAG represent at least a set of symptoms and a set of medical conditions, and wherein directed edges between the nodes represent relations between the nodes;
generating graph embeddings for the nodes of the DAG using a graph-embedding technique;
receiving an input indicative of a person's health wherein the input comprises a test set of symptoms;
extracting a set of test graph embeddings corresponding to the test set of symptoms from the graph embeddings; and
processing the test graph embeddings of the set using a neural network for obtaining a multi-label classification of the test set of symptoms matched to a test set of medical conditions as labels, wherein a given label is probabilistically indicative of presence of a medical condition.

In another aspect, an embodiment of the present disclosure provides an artificial intelligence (AI)-based system for medical diagnosis, the system comprising at least one processor, wherein the at least one processor is configured to:
obtain medical data from at least one data source, wherein the at least one data source is communicably coupled to the at least one processor;
generate a Directed Acyclic Graph (DAG) using the medical data, wherein nodes of the DAG represent at least a set of symptoms and a set of medical conditions, and wherein directed edges between the nodes represent relations between the nodes;
generate graph embeddings for the nodes of the DAG using a graph-embedding technique;
receive an input indicative of a person's health, wherein the input comprises a test set of symptoms;
extracting a set of test graph embeddings corresponding to the test set of symptoms from the graph embeddings; and
process the test graph embeddings of the set using a neural network for obtaining a multi-label classification of the test set of symptoms matched to a test set of medical conditions as labels, wherein a given label is probabilistically indicative of presence of a medical condition.

Embodiments of the present disclosure substantially eliminate or at least partially address the aforementioned problems in the prior art and enable accurate and efficient diagnosis of medical conditions.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 illustrates steps of an artificial intelligence (AI)-based method for medical diagnosis, in accordance with an embodiment of the present disclosure;

FIG. 2 illustrates an exemplary process flow of an artificial intelligence (AI)-based method for medical diagnosis, in accordance with an embodiment of the present disclosure;

FIG. 3 a schematic illustration of an artificial intelligence (AI)-based system for medical diagnosis, in accordance with an embodiment of the present disclosure;

FIG. 4 illustrates an exemplary process flow of a traditional machine learning model; and

FIG. 5 illustrates an exemplary process flow of a conventional deep learning model.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, an embodiment of the present disclosure provides an artificial intelligence (AI)-based method for medical diagnosis, the method comprising:
obtaining medical data from at least one data source;
generating a Directed Acyclic Graph (DAG) using the medical data, wherein nodes of the DAG represent at least a set of symptoms and a set of medical conditions, and wherein directed edges between the nodes represent relations between the nodes;
generating graph embeddings for the nodes of the DAG using a graph-embedding technique;
receiving an input indicative of a person's health, wherein the input comprises a test set of symptoms;
extracting a set of test graph embeddings corresponding to the test set of symptoms from the graph embeddings; and
processing the test graph embeddings of the set using a neural network for obtaining a multi-label classification of the test set of symptoms matched to a test set of medical conditions as labels, wherein a given label is probabilistically indicative of presence of a medical condition.

In another aspect, an embodiment of the present disclosure provides an artificial intelligence (AI)-based system for medical diagnosis, the system comprising at least one processor, wherein the at least one processor is configured to:
obtain medical data from at least one data source, wherein the at least one data source is communicably coupled to the at least one processor;
generate a Directed Acyclic Graph (DAG) using the medical data, wherein nodes of the DAG represent at least a set of symptoms and a set of medical conditions, and wherein directed edges between the nodes represent relations between the nodes;
generate graph embeddings for the nodes of the DAG using a graph-embedding technique;
receive an input indicative of a person's health, wherein the input comprises a test set of symptoms;
extracting a set of test graph embeddings corresponding to the test set of symptoms from the graph embeddings; and
process the test graph embeddings of the set using a neural network for obtaining a multi-label classification of the test set of symptoms matched to a test set of medical conditions as labels, wherein a given label is probabilistically indicative of presence of a medical condition.

The present disclosure provides the aforementioned artificial intelligence (AI)-based method and system for medical diagnosis. Herein, external factors (for example, demographic attributes, a set of medical history, a set of risk factors, and so forth), are included in the medical data and utilised to generate the DAG, such that representations of the DAG are learnt using these external factors as well. The method and system described in the present disclosure provides an accurate and efficient medical diagnosis, due to the inclusion of these external factors, and by accurately understanding relations between the symptoms and these external factors. Moreover, since the method and system described in the present disclosure utilise artificial intelligence techniques for learning representations in the DAG of at least the symptoms and medical conditions, they generate more meaningful representations of the graph embeddings, as compared to the presently available solutions. Such artificial intelligence techniques (namely, neural networks) are utilised for learning the relations between the set of symptoms and the set of medical conditions and processing the graph embeddings, whilst also considering the external factors. As a result, such graph embeddings provide higher accuracy in obtaining the multi-level classification and determining the presence of the medical condition. Utilising artificial intelligence technologies improves robustness and scalability of the aforementioned system and method. Moreover, the aforementioned system and method are also capable of incorporating real-time information and learning from it, so that any medical diagnosis provided is based on updated information.

It will be appreciated that the above-mentioned method and system are AI-based, which means that, the above-mentioned method and system employ AI techniques for providing the medical diagnosis. The AI techniques (such as deep learning techniques, machine learning techniques, and similar) are employed to learn relations between symptoms and medical conditions, and to process the graph embeddings for the medical diagnosis. Moreover, the term “medical diagnosis” refers to a determination of the medical condition, depending at least on symptoms experienced by a person. Medical conditions may also be dependent on other factors, which are described in detail herein later.

Throughout the present disclosure, the term “medical data” refers to a collection of information relating to symptoms, diagnosis, care and treatment of persons. Optionally, the medical data is at least one of: structured medical data, unstructured medical data. Herein, the structured medical data refers to medical data which is specific (namely, definitive) in nature and stored in a pre-defined format. Optionally, the structured medical data is implemented as at least one of: a tabular dataset, an Electronic Health Record (EHR), a HyperText Markup Language (HTML) dataset, a Structured Query Language (SQL) dataset. Other forms of the structured medical data are also feasible. Moreover, the unstructured medical data refers to medical data which includes varied types of data stored in native formats. Optionally, the unstructured medical data is implemented as at least one of: raw text in documents, articles, or medical journals; a recording of consultation sessions, surgeries or simulated procedures; a digital image of a diagnostic scan. Other forms of the unstructured medical data are also feasible.

Optionally, the medical data comprises at least one of: patient consultation data, patient diagnostic data, patient monitoring data, patient medical history data, patient data from health-related studies. Herein, the term “patient” refers to a person who requires medical treatment. Notably, the person may be considered to be a patient when the person has already received medical treatment or will be receiving medical treatment. The person may not be considered to be a patient if the person does not require medical treatment.

The patient consultation data refers to data pertaining to a consultation received by at least one patient from a medical professional. The patient diagnostic data refers to data pertaining to a diagnostic test of the at least one patient. The patient monitoring data refers to data pertaining to monitoring of vital signs (for example, heart rate, oxygen level, blood pressure, and so forth) of the at least one patient and/or a progress of the at least one patient during recovery from some medical condition. The patient medical history data refers to data pertaining to a historical record of health of the at least one patient. Optionally, the patient medical history data also comprises family medical history of the at least one patient. Beneficially, such medical data provides an improved insight of health of the person, since many medical conditions may occur due to a genetic predisposition. The patient data from health-related studies refers to data pertaining to health of the at least one person before, during and/or after participating in the health-related studies.

As an example, the medical data of a given patient may comprise at least one of: a personal information pertaining to the at least one patient, a symptom experienced by the at least one patient, a medical advice provided by at least one medical professional, a test conducted on the at least one patient, a diagnosis provided to the at least one patient, a medical prescription provided to the at least one patient, a medicine administered to the at least one patient, a medical treatment provided to the at least one patient, a surgery undergone by the at least one patient. In this regard, the personal information comprises, but is not limited to, a name, an age, a gender, an address, and an allergy.

Such medical data is very comprehensive and includes not just actual medical information, but also auxiliary information related to external factors which may affect health of the at least one patient. It will be appreciated that the medical data is bulk data, optionally having health-related data from a plurality of persons and optionally captured over a vast period of time. Beneficially, such medical data provides accurate analytical insight regarding health of the at least one patient, commonly experienced symptoms, regularly occurring medical conditions (for example, depending on external factors such as a geographical area, gender, age group, and so forth), common factors for certain medical conditions (for example, unhealthy eating habits may be caused because of increased stress), and the like. Such analytical insight assists the system in accurately identifying presence of a given medical condition.

Optionally, the medical data comprises at least one natural-language description, and wherein the method further comprises pre-processing the medical data prior to the step of generating the DAG, by processing the natural-language description using a phrase matching technique to generate a probable list of the set of symptoms, said probable list being used subsequently for generating the DAG. The term “natural-language description” refers to a description of the medical data in a naturally evolved language. Natural-language descriptions are commonly provided by people as they typically converse in their natural language. In a first example, natural language description of itchy eyes may be: itching in my eye, allergy in my eye, scratchy eye, irritation in my eye, and so forth. In a second example, natural language description of heartburn may be: burning in stomach, burning pain in stomach, acidic taste in my mouth with stomach-ache, and so forth. It will be appreciated that the set of symptoms are represented in the DAG using appropriate medical jargon (for example, itchy eyes, heartburn, and the like). Notably, a given natural language description may be transformed to a plurality of medical jargons, and vice versa.

Optionally, the pre-processing of the medical data is performed using a transformer-model architecture. The transformer-model architecture refers to an architecture which transforms (i.e., processes) the natural-language description to the probable list of the set of symptoms in medical jargon (namely, medical terminology). Optionally, the transformer-model architecture comprises: an encoder, a decoder. Herein, the encoder encodes the natural-language description to obtain a computer-readable description, and the decoder decodes the computer-readable description to the medical jargon.

The phrase matching technique refers to a technique which semantically matches a phrase having a natural-language description to a corresponding phrase having a medical jargon. Multiple natural-language descriptions can have a same corresponding medical jargon, and vice versa. In accordance with the first example, the natural-language descriptions (i.e., phrases): itching in my eye, allergy in my eye, scratchy eye, and irritation in my eye, are matched with the medical jargon ‘itchy eye’. In accordance with the second example, the natural-language descriptions: burning in stomach, burning pain in stomach, and acidic taste in my mouth with stomach-ache, are matched with the given medical jargon ‘heartburn’. Herein, the medical jargon is utilised to generate the probable list of the set of symptoms. In accordance with the first example and the second example, the probable list of the set of symptoms may include itchy eye and heartburn. The phrase matching algorithm inputs the at least one natural-language description and outputs best-matching symptoms in medical jargon. Since common people (i.e., people who do not have medical knowledge) cannot be expected to be familiar with accurate medical jargons, they may provide inputs (constituting the medical data) in the form of the natural-language descriptions. Thus, such natural-language descriptions are transformed to generate the probable list of the set of symptoms using the medical jargons. Optionally, the phrase matching technique is employed using a phrase matching model. A technical benefit of pre-processing the medical data is that this effectively tags and identifies symptoms being experienced by the person as medical jargons, ultimately increasing the efficiency and accuracy of the medical diagnosis.

Throughout the present disclosure, the term “data source” refers to a data repository wherein at least the medical data is stored. In an embodiment, the medical data is pre-generated and is pre-stored at the data source. In another embodiment, the medical data is generated by the at least one processor and is stored at the data source.

Optionally, the step of generation of the DAG comprises building a directed graph without any cycles. Throughout the present disclosure, the term “Directed Acyclic Graph (DAG)” refers to a graphical representation of a series of nodes arranged in an order, wherein at least two nodes are connected by a directed edge (i.e., a line). Herein, a given node represents an entity (i.e., a symptom, a medical condition, and the like) in the DAG. The term “directed edge” refers to an edge having a defined direction, such that each edge in the DAG represents a single directional flow from one node to another. Herein, a direction and length of the edge represents the relations between the nodes. Optionally, greater the length between two nodes, weaker the relation between the same, and vice versa. It will be appreciated that in the DAG, if a given directed edge is followed from a first node to a second node, there is no path to return to the first node (since the DAG is acyclic and does not have any loops or cycles). Optionally, the DAG is a multi-partite graph. The multi-partite graph represents multiple disjoint sets, wherein a given edge connects a first represented node from a first disjoint set to a second represented node from a second disjoint set. It will be appreciated that an edge does not connect two represented nodes from a same disjoint set. In an example, if the first node represents tooth-ache (from the set of symptoms) and the second node represents tooth sensitivity (from the set of medical conditions), such that the directional flow of the directed edge is from the first node to the second node, the directed edge may represent that tooth-ache is a symptom of tooth sensitivity. Moreover, the set of symptoms includes all possible symptoms experienced by persons and the set of medical conditions includes all possible medical conditions experienced by persons.

Optionally, different types of nodes are represented differently in the DAG. For example, the nodes representing the set of symptoms may be represented differently from the nodes representing the set of medical conditions. Optionally, the different types of nodes are represented differently implementing at least one of: a different colour for each type of node, a different size for each type of nodes, a different shape for each type of nodes, a different pattern for each type of nodes. In pursuance to the above example, the nodes representing the set of symptoms may be represented using the colour ‘pink’, and the nodes representing the set of medical conditions may be represented using the colour ‘green’.

Optionally, the nodes of the DAG also represent at least one of: a set of demographic attributes, a set of medical history, a set of risk factors. Such additional factors (i.e., demographic attributes, medical history, risk factors) may be considered as external factors, and are often responsible for symptoms being experienced by the person, having a direct impact on the medical diagnosis. The term “demographic attributes” refers to general characteristics of the persons. Examples of demographic attributes include, but are not limited to, an age, a gender, a race, an employment type, and a geographic location. The term “medical history” refers to a historic record pertaining to the person's health. Optionally, the medical history comprises the family medical history. Examples of medical history include, but are not limited to information pertaining to: an allergy, an illness, a surgery, an immunization, a result of a physical exams and/or test, a health habit, a pre-existing medical condition, and an ongoing treatment. The term “risk factors” refer to determinants associated with an increased risk of a medical condition. Optionally, the risk factors depend on at least one of: a genetic predisposition, an aspect of personal behaviour, a lifestyle habit, an environmental exposure. A technical advantage of the DAG also representing the above is that such additional factors provide a more accurate medical diagnosis, as compared to when these additional factors are not considered. These factors enable an indication of relations between symptoms and medical conditions, such that they may not only be responsible for the symptoms, but they also impact the medical diagnosis. Such additional factors assist in accurately relating symptoms with appropriate medical conditions. Moreover, representing such additional factors expands the DAG to be information rich and more meaningful, making it extensively robust and scalable.

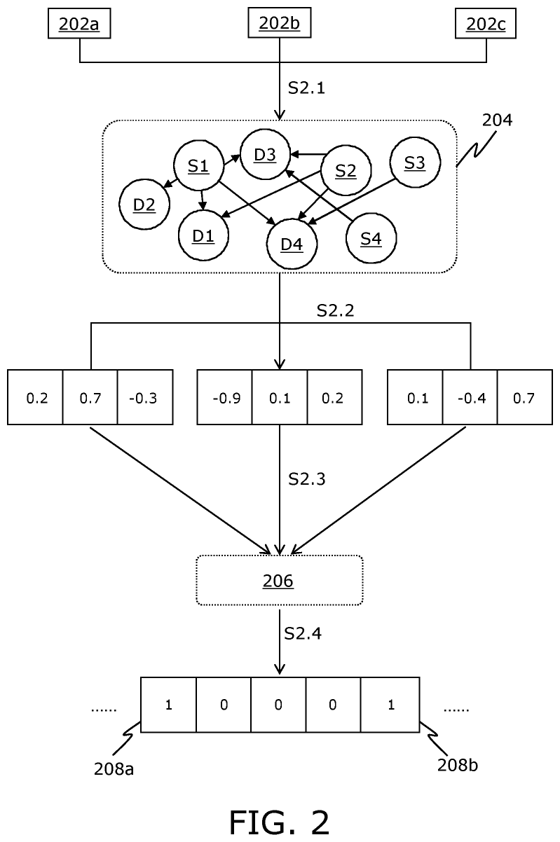

Throughout the present disclosure, the term “graph embeddings” refer to dense vector representations of the nodes of the DAG, which are learnt using graphs instead of textual sentences or paragraphs. Herein, when the nodes highly relate to each other, they are embedded close to each other and when the nodes weakly relate or do not relate to each other, they are embedded far from each other. For example, a first graph embedding representing a first symptom ‘arm pain’ may be embedded closer to a second graph embedding representing a second symptom ‘arm stiffness’, whereas a third graph embedding representing a third symptom ‘leg pain’ may be embedded farther away from the first graph embedding and the second graph embedding. Optionally, the graph embeddings are also stored at the data source. It will be appreciated that such graph embeddings are dense vectors (i.e., vectors majorly having non-zero values) having an arbitrary size (i.e., they are n-dimensional). Moreover, numbers represented in such graph embeddings (as shown in FIG. 2) are random descriptions of the dense vector(s). For example, the graph embedding of a dense vector may be 700-dimensional, and be represented by [ . . . , 0.2, −0.07, 0.9, . . . ]

The term “graph-embedding technique” refers to a technique which enables transformation of nodes, edges, and features of the DAG into vector space (i.e., a lower dimension) whilst maximally preserving properties (i.e., graph structure, information, and so forth). Notably, the graph embeddings are generated by learning a representation of a given node by considering neighbouring nodes of the given node as context (i.e., connectivity, structural role, node features, and the like). For example, when learning representation of a given node whose neighbouring nodes are: arm pain, arm swelling, arm stiffness, it may be determined that the given node is related to an arm with a high amount of discomfort and pain.

Optionally, the graph-embedding technique is implemented as at least one of: a vertex embedding technique, a word-to-vector embedding technique, a graph-to-vector embedding technique. The vertex embedding technique is a technique wherein each node (i.e., vertex) is encoded with its own vector representation. This technique is beneficial for predictions and visualisations on a nodal (i.e., vertex) level, since it is based on identifying similarities and dissimilarities between the nodes. Optionally, the vertex embedding technique is implemented as at least one of: a DeepWalk embedding technique, a node-to-vector (Node2vec) embedding technique, a structural deep network embedding technique. Such graph embedding techniques are well known in the art. A technical advantage of implementing the graph-embedding technique as one of the above is that these techniques generate meaningful representations of at least symptoms and medical conditions (having dense representations and relations therein), which are then used for obtaining the labels (and therefore, the medical diagnosis) corresponding to such symptoms and medical conditions. Moreover, for generating graph embeddings of a given node, initial vector representations of the given node represent one of: a random vector, a pre-existing dense representation.

The term “input” refers to information provided by the user, wherein the information pertains to symptoms being experienced by the person at a given time. For example, the input may be: headache, nausea. Such input may be received from a given user device. Moreover, the input is provided to the given user device by at least one of: the person, a medical practitioner. Such input is optionally processed to identify the test set of symptoms. It will be appreciated that the input is utilised for deriving inference from the graph embeddings for the test set of symptoms.

Optionally, the input is in form of a given natural-language description, and wherein the method further comprises processing the given natural-language description using a phrase matching technique to generate the test set of symptoms corresponding to the person's health. The input is in form of the given natural-language description since the person may not know appropriate medical jargon. Herein, the phrase matching technique is used to identify the medical jargon (i.e., during inference), from the input, since the pre-generated graph embeddings are expressed using medical jargon. In a first example, the given natural-language description of the input may be high body temperature, which when processed, may provide the test set of symptoms as fever. A technical benefit of the input being in the given natural-language description is this that the person may not know the appropriate medical jargon. Processing the given natural-language description to generate the test set of symptoms ensures that a correct correlation is formed between the natural-language description of the input and the graph-embeddings.

It will be appreciated that the test set of graph embeddings pertain to a finite number of graph embeddings, wherein each graph embedding in the test set of graph embeddings corresponds to each symptom in the test set of symptoms. This means that the test set of graph embeddings are a subset of the graph embeddings. For example, there may be 5000 graph embeddings existing, but for a given case, there may only be 10 graph embeddings in the test set of graph embeddings. Moreover, the test set of graph embeddings are extracted for deriving inference from the graph embeddings for the test set of symptoms. As per the first example, graph-embeddings corresponding to ‘fever’ may be extracted to obtain the test set of symptoms (i.e., other symptoms similar to fever, that the person may be experiencing) and thereafter, the test set of medical conditions (i.e., medical conditions which are indicated by fever, for example, covid, viral, malaria, and the like). Subsequently, the test graph embeddings are processed to obtain the multi-label classification.

The term “neural network” refers to a network of interconnected nodes, which mimic interconnections of neurons in a human brain. Examples of the neural network include, but are not limited to, a Feedforward Neural Network, a Radial basis function Neural Network, a Kohonen Self Organizing Neural Network, a Recurrent Neural Network (RNN), a Convolutional Neural Network, and a Modular Neural Network.

Beneficially, the neural network recognizes patterns and correlations in the graph embeddings for the nodes of the DAG, cluster such patterns and correlations, and classify them to correspond to at least one medical condition with certain probability. As the neural network processes an increasing amount of information, the neural network continuously learns and improves (i.e., due to hierarchical memory, which is constantly updating).

The test set of symptoms refers to symptoms being experienced by the person at a given time, and the test set of medical conditions refers to possible medical conditions that the person may be experiencing at the given time, in congruence with the test set of symptoms. Moreover, the term “multi-label classification” refers to a classification wherein two or more labels are assigned to a given instance. Herein, the given instance pertains to a given test set of symptoms (for example, such as, by a user). As an example, the given test set of symptoms is such: irritable throat, wet cough, runny nose, pain in throat, low temperature, chest congestion. Furthermore, the graph embeddings generated for the nodes of the given DAG may be: [ . . . . 0.2, −0.7, 0.3], [ . . . . −0.9, 0.1, 0.2. . . . ], [ . . . . 0.1, 0.4, −0.7 . . . . ], [ . . . . 0.84, −0.93, 0.19 . . . . ], [ . . . . −0.91, 0.5, 0.67 . . . . ], [ . . . . 0.27, 0.32, −0.16 . . . . ]. These graph embeddings, when processed using the neural network, may provide the multi-label classification as: cold (0.83), cough (0.72), and tonsils (0.54), wherein the classification has three labels, which means that there is a probability of presence of three separate medical conditions (i.e., the test set of medical conditions) based on the aforesaid set of six symptoms. In this example, a probability of cold may be 83%, a probability of cough may be 72%, and a probability of tonsils may be 54%. In continuation of this example, the given DAG may also include nodes representing external factors such as weather change, age of 40, medical history of pneumonia. When these external factors are considered, the multi-label classification may be updated as: cold (0.92), cough (0.55), tonsils (0.64) and pneumonia (0.54), which means that there is a probability of presence of four separate medical conditions based on the aforesaid symptoms and external factors. In this example, a probability of cold may be 92%, a probability of cough may be 55%, a probability of tonsils may be 64%, and a probability of pneumonia may be 54%. It will be appreciated that the input is processed for obtaining the test set of symptoms and the test set of medical conditions.

Optionally, the step of processing the graph embeddings using the neural network comprises:
converting vector representations in the graph embeddings into probabilities of presence of all medical condition(s) amongst the set of medical conditions, given the set of symptoms; and
generating the multi-label classification of the set of symptoms using the probabilities.

Optionally, a number of labels in the multi-label classification is equal to a number of medical conditions that are likely to be present, from amongst the set of medical conditions. The set of medical conditions may have hundreds of such medical conditions, but only a few might be feasible for a given set of symptoms. So, the multi-label classification has only as many labels as feasible medical conditions for the given set of symptoms, and each label has a probability of occurrence corresponding to each medical condition. It will be appreciated that the number of labels in the multi-label classification may change with a change in factors of the medical data. Moreover, graph embeddings of all symptoms of the test set of symptoms are combined together to form a single dense vector. Herein, the graph embeddings are combined by using at least one of: an averaging technique, a summing technique, a weighted sum technique, a weighted mean technique. Optionally, the vector representations in the graph embeddings are converted using a sigmoid activation function. The term “sigmoid activation function” refers to an activation function which is used for neural networks. An output from the sigmoid activation function is a raw probability that lies between 0 and 1. Herein, the sigmoid activation function converts arbitrary values of the vector representations into probabilities for generating the multi-label classification. The multi-label classification provides probabilities of occurrence of multiple medical conditions, along with names, identifiers, or similar of the medical conditions. Such probability values assist in ascertaining the medical diagnosis accurately. In the above example, values of cold (0.92), cough (0.55), tonsils (0.64) and pneumonia (0.54) are determined using the sigmoid activation function. A technical advantage of processing the graph embeddings by following the above-mentioned steps is this that it ensures that the multi-label classification provides significant improvement in accuracy of the medical diagnosis.

Optionally, the method further comprises:
generating a visualisation of the graph embeddings in a latent space; and
presenting the visualisation of the graph embeddings onto a user interface of a user device.

Herein, the visualisation is a visual representation of the graph embeddings (of the nodes of the DAG). Moreover, the latent space refers to a mathematical space in which the graph embeddings are present. Optionally, the visualisation is implemented at least as a t-Distributed Stochastic Neighbor Embedding (t-SNE) plot. The t-SNE plot is a statistical plot for visualizing the graph embeddings (high-dimensional data) by providing each graph embedding a location in an N-dimensional map. Optionally, the user interface is interactive, such that the user can interact with the visualisation. Herein, the user may interact with the visualisation in many ways, for example, zooming the visualisation to see relations between different graph embeddings, decide and select graph embeddings, comparing results of the visualisation with prior technology, and the like. Optionally, the user device is communicably coupled to the at least one processor. Examples of the user device include, but are not limited to, a mobile phone, a computer, a laptop, and a smartwatch. It will be appreciated that the visualisation provides additional information and support to the user, and assists in correlating the set of symptoms with the medical diagnosis. A technical advantage of generating and presenting the visualisation on the user device is that it provides an accurate correlation between the nodes of the DAG to a user, since it indicates how similar or dissimilar entities in the visualisation are. For example, similar entities will be clustered together, and vice versa. Herein, the user may be any person (such as the at least one patient, a medical professional, and the like).

The present disclosure also relates to the system as described above. Various embodiments and variants disclosed above, with respect to the aforementioned first aspect, apply mutatis mutandis to the system.

Optionally, the at least one data source is implemented as at least one of: a data source of the system, an external data source. In a case, when the at least one data source is implemented as the data source of the system, data pertaining to the system (for example, such as the medical data, the DAG, the graph embeddings, the multi-label classification, the visualisation, and the like) is stored at the data source of the system and accessed by the at least one processor. In another case, when the at least one data source is implemented as the external data source, data pertaining to the system (for example, such as the medical data, the DAG, the graph embeddings, the multi-label classification, the visualisation, and the like) may be stored at the external data source and may be accessed by the at least one processor.

Optionally, the medical data comprises at least one of: patient consultation data, patient diagnostic data, patient monitoring data, patient medical history data, patient data from health-related studies.

Optionally, the medical data comprises at least one natural-language description, and wherein the at least one processor is further configured to pre-process the medical data prior to the step of generating the DAG, by processing the at least one natural-language description using a phrase matching technique to generate a probable list of the set of symptoms, said probable list being used subsequently for generating the DAG.

Optionally, the input is in form of a given natural-language description, and wherein the at least one processor is further configured to process the given natural-language description using a phrase matching technique to generate the test set of symptoms corresponding to the person's health.

Optionally, the nodes of the DAG also represent at least one of: a set of demographic attributes, a set of medical history, a set of risk factors.

Optionally, the graph-embedding technique is implemented as at least one of: a vertex embedding technique, a word-to-vector embedding technique, a structural deep network embedding technique, and a graph-to-vector embedding technique.

Optionally, when processing the graph embeddings using the neural network, the at least one processor is configured to:
convert vector representations in the graph embeddings into probabilities of presence of all medical condition(s) amongst the set of medical conditions, given the set of symptoms; and
generate the multi-label classification of the set of symptoms using the probabilities.

Optionally, the at least one processor is further configured to:
generate a visualisation of the graph embeddings in a latent space; and
present the visualisation of the graph embeddings onto a user interface of a user device, wherein the user device is communicably coupled to the at least one processor.

### Experimental Part

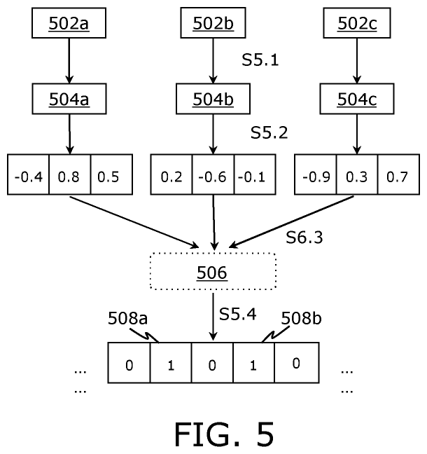

A test simulation of the above-mentioned AI-based system for medical diagnosis was conducted. This test simulation compared the above-mentioned system (which implements the aforementioned method) with presently-existing technologies of a traditional machine learning model and a conventional deep learning model. This test simulation consisted of multiple experiments conducted using medical data sourced from various sources including Centre for Disease Control and Prevention (CDC), Unified Medical Language System (UMLS), and various other biomedical and healthcare websites regarding symptom-medical condition interactions, definitions, demographic-risk factors, and the like. A detailed description of the processes followed by these systems are provided hereinbelow in FIGS. 2, 4 and 5. The process flow of the AI-based system for medical diagnosis as described in the present disclosure provided in FIG. 2, the process flow followed by the traditional machine learning model is provided in FIG. 4 and the process flow followed by the conventional deep learning model is provided in FIG. 5.

The medical data was provided to all of these systems, with a same set of symptoms: Nausea, Headache and Heartburn. Probable medical conditions identified by these systems were expressed in multi-label classifications with corresponding sets of medical conditions as labels. These multi-label classifications were then evaluated using the macro-averaged F1 score (Macro-F1 score) to assess an accuracy of the multi-label classifications generated by these systems.

It was observed that the traditional machine learning model had an accuracy of 85%, which was considered the baseline for this test simulation. Moreover, it was observed that the conventional deep learning model provided an improvement compared to the traditional machine learning model, with an accuracy of 92% (i.e., an improvement of approximately 7% over the baseline). Furthermore, it was observed that the AI-based system of the present disclosure provided a significant improvement over both of these prior systems, with an accuracy of 97.5% (i.e., an improvement of approximately 12.5% over the baseline), when only symptoms and medical conditions were included in the DAG. However, when the DAG was appended with external factors (for example, such as, tests, medications, demographic information, risk-factors, and so forth), an additional 2% improvement was observed in overall diagnostic performance, with the AI-based system of the present disclosure having an accuracy of 99.5% (i.e., approximately 14% over the baseline). This test simulation proved that the AI-based system and method for medical diagnosis described herein are much better than any presently existing systems and methods for medical diagnosis.

### DETAILED DESCRIPTION OF THE DRAWINGS

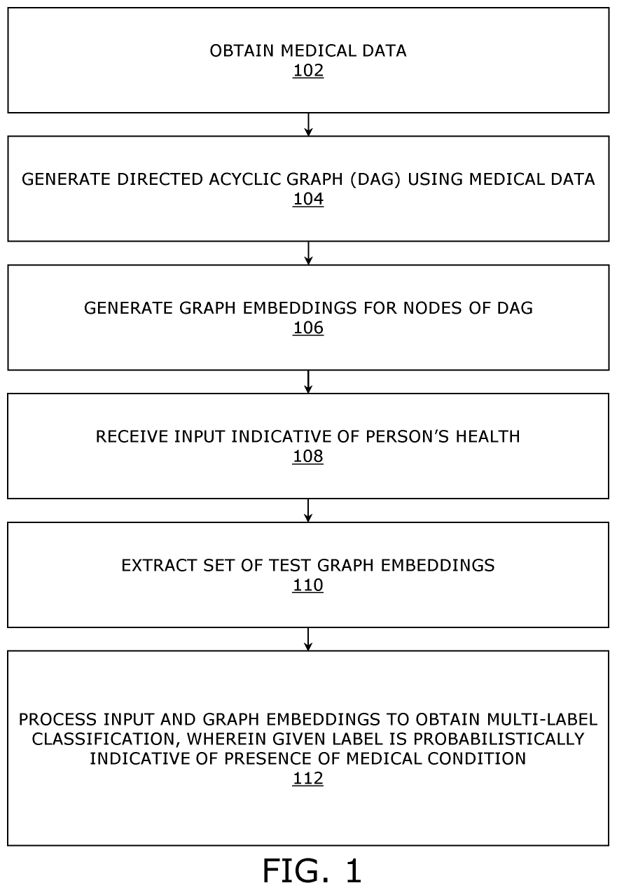

Referring to FIG. 1, illustrated are steps of an artificial intelligence (AI)-based method for medical diagnosis, in accordance with an embodiment of the present disclosure. At step 102, medical data is obtained from at least one data source. At step 104, a Directed Acyclic Graph (DAG) is generated using the medical data, wherein nodes of the DAG represent at least a set of symptoms and a set of medical conditions, and wherein directed edges between the nodes represent relations between the nodes. At step 106, graph embeddings are generated for the nodes of the DAG using a graph-embedding technique. At step 108, an input indicative of the person's health is received, wherein the input comprises a test set of symptoms. At step 110, a set of test graph embeddings corresponding to the test set of symptoms are extracted from the graph embeddings. At step 112, the test graph embeddings of the set are processed using a neural network to obtain a multi-label classification of the test set of symptoms matched to the test set of medical conditions as labels, wherein a given label is probabilistically indicative of presence of a medical condition.

The aforementioned steps are only illustrative and other alternatives can also be provided where one or more steps are added, one or more steps are removed, or one or more steps are provided in a different sequence without departing from the scope of the claims herein.

Referring to FIG. 2, illustrated is an exemplary process flow of an artificial intelligence (AI)-based method for medical diagnosis, in accordance with an embodiment of the present disclosure. At S2.1, a test set of symptoms 202a, 202b, 202c (hereinafter collectively referred as 202) is obtained. A Directed Acyclic Graph (DAG) 204 is previously generated using medical data. Nodes of the DAG 204 represent at least a set of symptoms S1, S2, S3, S4 and a set of medical conditions (namely, diseases) D1, D2, D3, D4. Directed edges between the nodes represent relations between the nodes. Moreover, graph embeddings are previously generated for the nodes of the DAG 204 using a graph-embedding technique. At S2.2, the graph embeddings are extracted from stored data based on the test set of symptoms 202, and passed for further machine learning processes. Herein, numbers represented in the graph embeddings are random descriptions of dense vector(s) formed in the DAG. At S2.3, the graph embeddings are processed using a neural network 206. Herein, the graph embeddings are combined by using at least one of: an averaging technique, a summing technique, a weighted sum technique, a weighted mean technique. At S2.4, the combined graph embeddings are processed to obtain a multi-label classification. The test set of symptoms matched to the test set of medical conditions are represented as labels in the multi-label classification. Herein, labels 208a, 208b (hereinafter collectively referred as 208) are probabilistically indicative of presence of their corresponding medical conditions. Herein, probability represented in a given label is thereon compared with a threshold to identify presence of the corresponding medical condition. For example, the labels 208a and 208b have probability of 0.8 and 0.75, respectively, while all other labels (indicated in middle of 208a and 208b) have a probability of less than 0.5. If the threshold is set to be 0.7, only the labels 208a and 208b indicate presence, while the other labels indicate absence of their corresponding medical condition. Therefore, the multi-label classification may represent a presence of pulmonary embolism and gastritis.

The aforementioned process flow is only illustrative and other alternatives can also be provided where one or more steps are added, one or more steps are removed, or one or more steps are provided in a different sequence without departing from the scope of the claims herein.

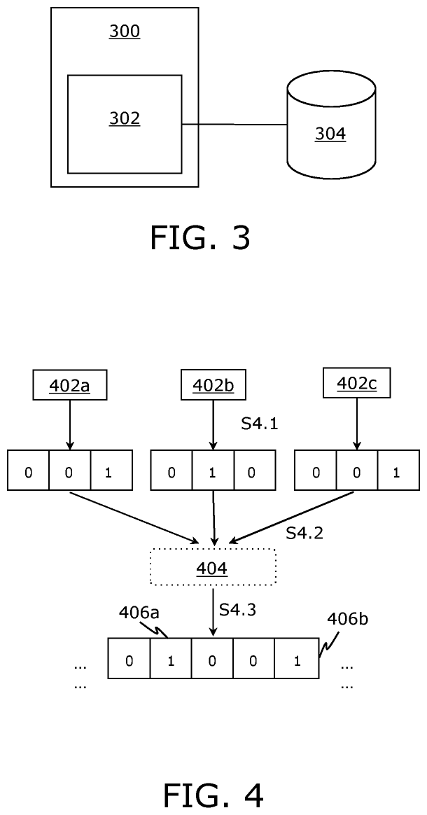

Referring to FIG. 3, illustrated is a schematic illustration of an artificial intelligence (AI)-based system 300 for medical diagnosis, in accordance with an embodiment of the present disclosure. The system 300 comprises at least one processor (depicted as a processor 302). The processor 302 is communicably coupled with at least one data source (depicted as a data source 304). At least the medical data is stored at the data source 304. In some implementations, the data source 304 may also be a part of the system 300.

Referring to FIG. 4, illustrated is an exemplary process flow of a traditional machine learning model. At S4.1, symptoms 402a, 402b, 402c (hereinafter collectively referred as 402) are obtained and corresponding one-hot encodings are generated. For example, symptom 402a may be Nausea, symptom 402b may be Headache and symptom 402c may be heartburn. At step S4.2, the one-hot encodings are sent to a machine learning model 404 for processing. At step S4.3, the machine learning model 404 processes the one-hot encodings to generate a set of probable medical conditions 406a, 406b (hereinafter collectively referred as 406). For example, probable medical condition 406a may be acid reflux, and probable medical condition 406b may be gastritis.

Referring to FIG. 5, illustrated is an exemplary process flow of a deep learning model. At S5.1, symptoms 502a, 502b, 502c (hereinafter collectively referred as 502) are obtained corresponding to definitions 504a, 504b, 504c (hereinafter collectively referred as 504) of the symptoms 502. For example, if symptom 502a is Nausea, the definition 504a may be ‘unsettling feeling in the stomach, usually accompanied by an urge to vomit’; if symptom 502b is Headache, the definition 504b may be ‘pain or ache anywhere in the head (sharp, piercing or pounding)’; and if symptom 502c is heartburn, the definition 504c may be ‘discomfort in/around belly or chest, may feel like burning or heat’. At step S5.2, the definitions 504 are processed to generate definition embeddings, which are context-aware language-model based dense representations generated using text from definitions. At step S5.3, the definition embeddings are sent to a machine learning model 506 for processing. At step S5.4, the machine learning model 506 processes the definition embeddings to generate a set of probable medical conditions 508a, 508b (hereinafter collectively referred as 508). For example, probable medical condition 508a may be acid reflux, and probable medical condition 508b may be influenza.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. An artificial intelligence (AI)-based method for medical diagnosis, the method comprising:
obtaining medical data from at least one data source;
generating a Directed Acyclic Graph (DAG) (204) using the medical data, wherein nodes of the DAG represent at least a set of symptoms (S1-S4) and a set of medical conditions (D1-D4), and wherein directed edges between the nodes represent relations between the nodes;
generating graph embeddings for the nodes of the DAG using a graph-embedding technique;
receiving an input indicative of a person's health, wherein the input comprises a test set of symptoms (202);
extracting a set of test graph embeddings corresponding to the test set of symptoms from the graph embeddings; and
processing the test graph embeddings of the set using a neural network (206) for obtaining a multi-label classification (208) of the test set of symptoms matched to a test set of medical conditions as labels, wherein a given label is probabilistically indicative of presence of a medical condition.

2. A method according to claim 1, wherein the medical data comprises at least one of: patient consultation data, patient diagnostic data, patient monitoring data, patient medical history data, patient data from health-related studies.

3. A method according to according to claim 1, wherein the medical data comprises at least one natural-language description, and wherein the method further comprises pre-processing the medical data prior to the step of generating the DAG (204), by processing the natural-language description using a phrase matching technique to generate a probable list of the set of symptoms (S1-S4), said probable list being used subsequently for generating the DAG.

4. A method according to claim 1, wherein the input is in form of a given natural-language description, and wherein the method further comprises processing the given natural-language description using a phrase matching technique to generate the test set of symptoms (202) corresponding to the person's health, wherein the symptoms in the test set of symptoms also belong to the set of symptoms (S1-S4).

5. A method according to claim 1, wherein the nodes of the DAG (204) also represent at least one of: a set of demographic attributes, a set of medical history, a set of risk factors.

6. A method according to claim 1, wherein the graph-embedding technique is technique is implemented as at least one of: a vertex embedding technique, a word-to-vector embedding technique, a graph-to-vector embedding technique.

7. A method according to claim 1, wherein the step of processing the graph embeddings using the neural network (206) comprises:
converting vector representations in the graph embeddings into probabilities of presence of all medical condition(s) amongst the set of medical conditions (D1-D4), given the set of symptoms (S1-S4); and
generating the multi-label classification (208) of the set of symptoms using the probabilities.

8. A method according to claim 1, wherein the method further comprises:
generating a visualisation of the graph embeddings in a latent space; and
presenting the visualisation of the graph embeddings onto a user interface of a user device.

9. An artificial intelligence (AI)-based system (300) for medical diagnosis, the system comprising at least one processor (302), wherein the at least one processor is configured to:
obtain medical data from at least one data source (304), wherein the at least one data source is communicably coupled to the at least one processor;
generate a Directed Acyclic Graph (DAG) (204) using the medical data, wherein nodes of the DAG represent at least a set of symptoms (S1-S4) and a set of medical conditions (D1-D4), and wherein directed edges between the nodes represent relations between the nodes;
generate graph embeddings for the nodes of the DAG using a graph-embedding technique;
receive an input indicative of a person's health, wherein the input comprises a test set of symptoms (202);
extracting a set of test graph embeddings corresponding to the test set of symptoms from the graph embeddings; and
process the test graph embeddings of the set using a neural network (206) for obtaining a multi-label classification (208) of the test set of symptoms matched to a test set of medical conditions as labels, wherein a given label is probabilistically indicative of presence of a medical condition.

10. A system (300) according to claim 9, wherein the at least one data source (304) is implemented as at least one of: a data source of the system, an external data source.

11. A system (300) according to claim 9, wherein the medical data comprises at least one of: patient consultation data, patient diagnostic data, patient monitoring data, patient medical history data, patient data from health-related studies.

12. A system (300) according to claim 9, wherein the medical data comprises at least one natural-language description, and wherein the at least one processor (302) is further configured to pre-process the medical data prior to the step of generating the DAG (204), by processing the natural-language description using a phrase matching technique to generate a probable list of the set of symptoms (S1-S4), said probable list being used subsequently for generating the DAG.

13. A system (300) according to claim 9, wherein the input is in form of a given natural-language description, and wherein the at least one processor (302) is further configured to process the given natural-language description using a phrase matching technique to generate the test set of symptoms (202) corresponding to the person's health.

14. A system (300) according to claim 9, wherein the nodes of the DAG (204) also represent at least one of: a set of demographic attributes, a set of medical history, a set of risk factors.

15. A system (300) according to claim 9, wherein the graph-embedding technique is implemented as at least one of: a vertex embedding technique, a word-to-vector embedding technique, a graph-to-vector embedding technique.

16. A system (300) according to claim 9, wherein when processing the graph embeddings using the neural network (206), the at least one processor (302) is configured to:
convert vector representations in the graph embeddings into probabilities of presence of all medical condition(s) amongst the set of medical conditions (D1-D4), given the set of symptoms (S1-S4); and
generate the multi-label classification (208) of the set of symptoms using the probabilities.

17. A system (300) according to claim 9, wherein the at least one processor (302) is further configured to:
generate a visualisation of the graph embeddings in a latent space; and
present the visualisation of the graph embeddings onto a user interface of a user device, wherein the user device is communicably coupled to the at least one processor.
